(19) United States
(12) Patent Application Publication
(10) Pub. No.: US 2014/0134385 A1
(43) Pub. Date: May 15, 2014

(54) Low-temperature Thermoplastic Plate with Velvet Layer

(71) Applicant: China Xinjiang Ke Lan Shuang Yi Medical Technology Stock Co., Ltd., Urumqi (CN)

(72) Inventors: Jun YI, Urumqi (CN); Xin Jun HEI, Urumqi (CN)

(73) Assignee: China Xinjiang Ke Lan Shuang Yi Medical Technology Stock Co., Ltd., Urumqi (CN)

(21) Appl. No.: 13/900,223

(22) Filed: May 22, 2013

(30) Foreign Application Priority Data
Nov. 11, 2012 (CN) 201220589761.1

(51) Int. Cl. B32B 5/26; D06N 7/00; A61F 2/82

(52) U.S. Cl. CPC B32B 5/26; A61F 2/82; D06N 7/0097
USPC 428/95

(57) ABSTRACT

A low-temperature thermoplastic plate with velvet layer comprises a low-temperature thermoplastic plate and a velvet layer, wherein the low-temperature thermoplastic plate has meshes, and wherein the velvet layer is fixed to the regions which are on the top of the low-temperature thermoplastic plate and outside the meshes. The low-temperature thermoplastic plate with velvet layer of the present invention with velvet layers attached to the top and bottom thereof has rational and compact structure. Thereby, the low-temperature thermoplastic plate with velvet layer of the present invention has high gas permeability, high sweat absorbance and high extensibility and is comfortable for users and easy to work. Besides, the low-temperature thermoplastic plate with velvet layer of the present invention can be mass-produced in high efficiency.

The current application claims a foreign priority to the patent application of China No. 201220589761.1 filed on Nov. 11, 2012.

### BACKGROUND OF THE INVENTION

1. Field of the Invention

The present invention relates to a low-temperature thermoplastic plate, particularly to a low-temperature thermoplastic plate with velvet layer.

2. Description of the Related Art

The low-temperature thermoplastic plate is a new medical material fabricated via physically and chemically processing specially-synthesized polyester, featuring easiness to soften at low temperature, high plasticity, lightweight, high strength, high workability, high X-ray permeability and biodegradability. Therefore, the low-temperature thermoplastic plate is widely used as the material of orthopedic splints used before or after surgical operation. In fabrication of an orthopedic splint, the low-temperature thermoplastic plate is surface-treated to remove the stickiness thereof. Next, the low-temperature thermoplastic plate is softened via immersed it in water at a temperature of 65-70° C. for 3 minutes. Next, the softened low-temperature thermoplastic plate is taken out from hot water and plastically placed on the region needing splinting. Thus, the region is splinted after the low-temperature thermoplastic plate has hardened. However, the conventional low-temperature thermoplastic plate has poor gas permeability and inferior sweat absorbance, discomforting the users wearing the splints made of it. Besides, the fabric-coated low-temperature thermoplastic plate has low extensibility. Therefore, the conventional low-temperature thermoplastic plates have the disadvantages of poor gas permeability, inferior sweat absorbance, and low extensibility.

### SUMMARY OF THE INVENTION

The primary objective of the present invention is to provide a low-temperature thermoplastic plate with velvet layer to effectively solve the problems of poor gas permeability, inferior sweat absorbance and low extensibility of the conventional low-temperature thermoplastic plates.

To achieve the abovementioned objective, the present invention proposes a low-temperature thermoplastic plate with velvet layer, which comprises a low-temperature thermoplastic plate and a velvet layer, wherein the low-temperature thermoplastic plate has meshes, and wherein the velvet layer is fixed to the regions which are on the top of the low-temperature thermoplastic plate and outside the meshes.

The abovementioned low-temperature thermoplastic plate with velvet layer of the present invention is further optimized or improved via fixing a velvet layer to the regions which are on the bottom of the low-temperature thermoplastic plate; and sizing the velvet layer to have a thickness of 0.1-1 mm.

The low-temperature thermoplastic plate with velvet layer of the present invention with velvet layers attached to the top and bottom thereof has rational and compact structure. Thereby, the low-temperature thermoplastic plate with velvet layer of the present invention has high gas permeability, high sweat absorbance and high extensibility and is comfortable for users and easy to work. Besides, the low-temperature thermoplastic plate with velvet layer of the present invention can be mass-produced in high efficiency.

### BRIEF DESCRIPTION OF THE DRAWINGS

FIG. 1 is a sectional view schematically showing a low-temperature thermoplastic plate with velvet layer according to one embodiment of the present invention.

### DETAILED DESCRIPTION OF THE INVENTION

The present invention would not be limited by the embodiments described below but will be realized according to the technical schemes of the present invention and practical requirements.

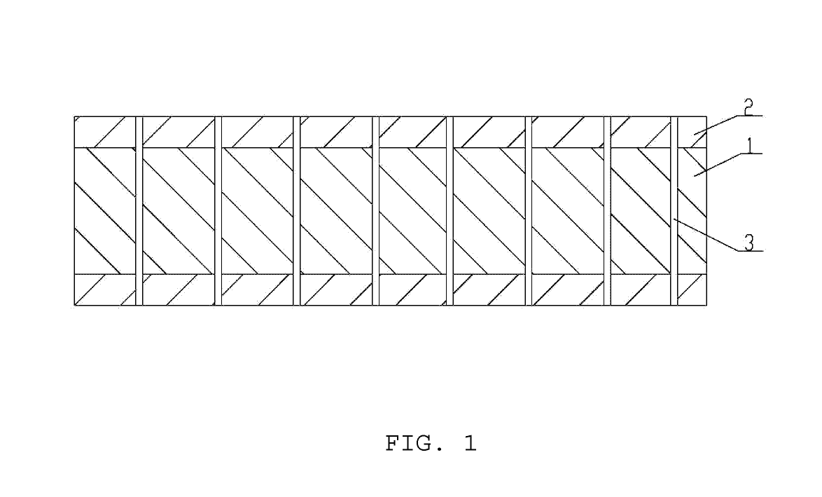

While reading the descriptions of relative positions mentioned in the specification of the present invention, such as front, rear, upper, lower, left, right, etc., the readers should refer to the structure shown in FIG. 1.

Below, the technical contents of the present invention are described in detail with the embodiments and the attached drawing.

Refer to FIG. 1. The low-temperature thermoplastic plate with velvet layer of the present invention comprises a low-temperature thermoplastic plate 1 and a velvet layer 2. The low-temperature thermoplastic plate 1 has meshes 3. The velvet layer 2 is fixed to the regions which are on the top of the low-temperature thermoplastic plate 1 and outside the meshes 3. The velvet layer 2 is fixed to the low-temperature thermoplastic plate 1 by electrostatic force. Therefore, the low-temperature thermoplastic plate with velvet layer of the present invention can be mass-produced in high efficiency. While users sweat, the velvet layer 2 can absorb sweat more effectively and thus exempt users from discomfort caused by wearing the splints made from the conventional thermoplastic plate. Further, the velvet layer 2 would not interfere with the extension of the low-temperature thermoplastic plate 1 in the present invention.

According to practical requirements, the low-temperature thermoplastic plate with velvet layer of the present invention is further optimized or improved via fixing a velvet layer 2 to the regions which are on the bottom of the low-temperature thermoplastic plate 1; and sizing the velvet layer 2 to have a thickness of 0.1-1 mm, as shown in FIG. 1.

The technical characteristics of the present invention have been described above. The embodiment of the present invention may involve all or part of the technical characteristics according to practical requirement.

What is claimed is:

1. A low-temperature thermoplastic plate with velvet layer, characterized in comprising a low-temperature thermoplastic plate and a velvet layer, wherein said low-temperature thermoplastic plate has meshes, and wherein said velvet layer is fixed to regions which are on a top of said low-temperature thermoplastic plate and outside said meshes.

2. The low-temperature thermoplastic plate with velvet layer according to claim 1, wherein a velvet layer is fixed to a bottom of said low-temperature thermoplastic plate.

3. The low-temperature thermoplastic plate with velvet layer according to claim 1, wherein said velvet layer has a thickness of 0.1-1 mm.
